(19) United States
(12) Patent Application Publication
(10) Pub. No.: US 2018/0000409 A1
(43) Pub. Date: Jan. 4, 2018

(54) Apparatus for the Assessment of the Level of Pain and Nociception During General Anesthesia Using Electroencephalogram, Plethysmographic Impedance Cardiography, Heart Rate Variability and the Concentration or Biophase of the Analgesics

(71) Applicants: Quantium Medical SL, Mataró, Barcelona (ES); Hospital Clinic de Barcelona, Barcelona (ES)

(72) Inventors: Erik Weber Jensen, Barcelona (ES); Luis Pedro Gambús, Barcelona (ES)

(21) Appl. No.: 15/536,591

(22) PCT Filed: Dec. 17, 2015
(86) PCT No.: PCT/DK2015/000052

§ 371 Date: Jun. 15, 2017

(30) Foreign Application Priority Data
Dec. 18, 2014 (DK) PA 2014 00742

(51) Int. Cl. A61B 5/00; A61B 5/0476; A61B 5/0456; A61B 5/0488; A61B 5/024

(52) U.S. Cl. CPC A61B 5/4821; A61B 5/0488; A61B 5/02405; A61B 5/0456; A61B 5/0476

(57) ABSTRACT

Means and methods for measuring pain and adapted for calculating the level of nociception during general anesthesia or sedation from data including electroencephalogram (EEG), facial electromyogram (EMG), heart rate variability (HRV) by electrocardiogram (ECG) and plethysmography by impedance cardiography (ICG). In a preferred embodiment of this invention the parameters derived from the EEG, the HRV, the plethysmographic curve and the analgetics concentrations are either combined into one index on a scale from 0 to 100, where a high number is associated with high probability of response to noxious stimuli, while a decreasing index is associated with decreasing probability of response to noxious stimuli. Zero (0) indicates extremely low probability of response to noxious stimuli. In an alternative embodiment, only features from the EEG and ECG will be used or only features from EEG, ECG and ICG, to define the fmal index.

### BACKGROUND OF THE INVENTION

Introduction to Anesthesia

Anesthesia has been defined as a drug induced state where the patient has lost consciousness, loss of sensation of pain or any other stimuli, i.e. analgesia, and amnesia of all the procedure. Furthermore the patient may be paralyzed as well. (Prys-Roberts C: Anesthesia: A practical or impossible construct? Br J Anaesth 1987; 59:1341-2). To obtain these objectives, the anesthesiologist can use different classes of drugs, mostly hypnotics and analgesics. This allow the patients to undergo surgery and other procedures without the distress and pain they would otherwise experience.

Continuously, the brain receives a vast amount of stimuli. However, the subject is only processing a small amount of it. When administering enough doses of hypnotics, the following loss of consciousness make that the patient does not perceive the stimuli, but the neurovegetative and somatic responses are not necessarily abolished. When administering enough doses of analgesics they block the nociceptive stimuli and prevent the neurovegetative and somatic responses. However they do not always produce a loss of consciousness and amnesia.

One of the objectives of modem anesthesia is to ensure adequate level of consciousness to prevent awareness without inadvertently overloading the patients with anesthetics which might cause increased postoperative complications. The overall incidence of intraoperative awareness with recall is about 0.2-2%, but it may be much higher in certain high risk patients like multiple trauma, caesarean section, cardiac surgery and hemodynamically unstable patients. Sometimes the patients could remember noises or even voices. In other cases the patients cannot move and advice to the anesthesiologist that they are awake or in the worst case in pain during the surgical maneuvers. This is a complication that can lead to severe postoperative psychosomatic dysfunction in almost 50% of patients. (Schwender D, Kunze-Kronawitter H, Dietrich P, Klasing S, Forst H, Madler C. Conscious awareness during general anesthesia: patients' perceptions, emotions, cognition and reactions. Br J Anaesth 1998; 80:133-39). As intraoperative awareness could be a major medico-legal liability to the anesthesiologists and could lead to postoperative psychosomatic dysfunction in the patients, it should therefore be avoided. (Sandin R H, Enlund G, Samuelsson P, Lennmarken C: Awareness during anesthesia: a prospective case study. Lancet. 2000; 355:707-11. Sandin RH. Awareness 1960-2002, explicit recall of events during general anesthesia. Adv Exp Med Biol. 2003; 523:135-47).

A usual clinical method for assessing the nociception during general anesthesia is the Ramsay scale where level 6 is no response to noxious stimulation.

Introduction to Analgesia

Nociception and the perception of pain define the need for analgesia, obtaining pain relief. The state of analgesia for surgery is reached by the administration of analgesics. The demand of analgesics is individual for each patient. Therefore there is a need for continuous preferably noninvasive monitoring of the analgesia of the patient. Autonomic responses such as tachycardia, hypertension, emotional sweating and lacrimation although non-specific are regarded as signs of nociception and consequently inadequate analgesia.

A method for monitoring skin conductance have been described in U.S. Pat. No. 6,571.124 “Apparatus and method for monitoring skin conductance and method for controlling a warning signal”, however this patent is intended for use with babies and does not take a multiparameter approach as in the present patent. Additionally, the method described in this patent applies a more extended analysis of the ICG by analyzing data with a Hilbert transform.

The autonomic nerve system (ANS) consists of two branches, the parasympathetic and the sympathetic system, where a sympathetic response is activated during nociception. A U.S. Pat. No. 7,024,234 “Method and apparatus for monitoring the autonomic nervous system” describes an algorithm that analyzes a photoplethysmographic signal for the detection of ANS activity during sleep related breathing disorders.

A method for assessing the level of nociception during anesthesia has been described in U.S. pat No. 2005143665 “Method and an apparatus for pulse plethysmograph based detection of nociception during anesthesia or sedation” by HUIKU MATTI V T (FI); KORHONEN ILKKA (FI); VAN GILS MARCUS J (FI); JOUSI MIKKO 0 (FI); LOTJONEN JYRKI M P (FI). This patent explores the detection of nociception by plethysmography (PM) from which a number of parameters are derived: the max amplitude of the PM curve, the amplitude of the dicrotic notch, the Heart Beat interval (RR) and other parameters from the PM curve. These parameters are used to design a final index using a multiple logistic regression approach.

In the present patent a combination of additional parameters of HR, SpO2, ICG, EEG and facial EMG is applied. This gives a safer method because the final index, termed “Composite Nociception Index” (CNI), is a combination of parameters which are independent.

In 1992 Pomfrett filed the patent application for U.S. Pat. No. 5,372,140 which proposes a method and an apparatus for providing a measure of the depth of anesthesia based on analyzing beat-to-beat heart rate together with respiration. For that purpose a series of so-called R-waves from the cardiac signal is analyzed for determining the position in time of each R-wave relative to the respiratory cycle within which it occurs, and a measurement value representing the degree of clustering is derived. Further a so-called circular statistics is utilized with a test for randomness, and finally the measurement value is compared with a reference value to find the depth of anesthesia. The proposed measure is related to respiratory sinus arrhythmia, which is primarily controlled by parasympathetic nervous system. The measure is poorly related to the functioning of the sympathetic nervous system, and hence may not measure sympathetic reactions to pain. As a conclusion, the disclosed method and apparatus does not provide results, which could be considered as an objective measure for the level of analgesia of a patient.

Cohen-laroque filed in 1998 the patent application “Device for determining the depth of anesthesia”, Document U.S. Pat. No. 6,120,443. The patent describes a cardiac activity of the patient, detecting a periodic wave there from, calculating time intervals between successive waves, determining a series of time intervals, and calculating a fractal dimension of said series as well as calculating a depth of anesthesia as a function of the fractal dimension. The signal is filtered by calculating the maximum correlation between the sampled signal and a theoretical signal, whereupon a signal period between 20 and 80 intervals is suggested, then regrouping the time intervals to form several digital series, and finally the fractal dimension is approximated by determining a dimension of correlation between said digital series, whereupon two to ten series is used. The disclosed method is mathematically based on computing correlation dimension for beat-to-beat heart rate signal. Theoretically the correlation dimension requires a very long data sequence, leading to large delays in real-time monitoring. Though the inventor proposes to use relatively short data sequences to minimize the acquisition delay this makes the theoretical basis of the method questionable. As above, also this disclosed method and apparatus does not provide results, which could be considered as an objective or reliable measure for the level of analgesia of a patient.

Huiku et al filed 14/10/2002 “A method and an apparatus for pulse plethysmograph based detection of nociception during anesthesia or sedation” and in 2003 Iika Korhonen filed an application termed “Monitoring a condition of a patient during anesthesia or sedation” U.S. Pat. No. 6,685,649. Both patents refer to an invention for detection of nociception by analysis of RR intervals achieved either from the ECG or from the BP. From the RR interval the acceleration emphasized RR interval is defined. The patent is different from the present invention as it does not combine the ECG with the features extracted from EEG nor does it use the analgetics concentrations.

In another example, patent document U.S. Pat. No. 5,372,140 recites a method and an apparatus for providing a measure of the depth of anesthesia based on analyzing beat-to-beat heart rate together with respiration, which does not provide results that could be considered as an objective measure for the level of analgesia of a patient. Patent document U.S. Pat. No. 6,120,443 recites a method for calculating the depth of anesthesia as a function of the fractal dimension of a series of time intervals between successive waves of cardiac activity of a patient. This disclosed method is mathematically based on computing correlation dimension for beat-to-beat heart rate signal. Theoretically, the correlation dimension requires a very long data sequence, which leads to large delays in real-time monitoring.

Nociception and the perception of pain define the need for analgesia for obtaining pain relief. The state of analgesia for surgery is reached by the administration of analgesics. The demand of analgesics is individual for each patient, therefore, there is a need for continuous, preferably non-invasive, monitoring of the analgesia of the patient. Autonomic responses such as tachycardia, hypertension, emotional sweating and lacrimation, although non-specific, are regarded as signs of nociception and consequently inadequate analgesia.

Several methods for monitoring nociception were previously recited. A monitoring method by skin conductance has been claimed in patent document U.S. Pat. No. 6,571,124, however, this method is specified for use solely with neonates and does not take a multi-parameter approach. In another example, patent document U.S. Pat. No. 7,024,234 recites an algorithm that analyzes a photoplethysmographic signal for the detection of the autonomic nervous system activity during sleep related breathing disorders. In yet another example, patent document US 2005143665 recites a method for assessing the level of nociception during anesthesia by plethysmography from which a number of parameters are derived which are used to design a final index using a multiple logistic regression approach. In yet another example, patent document 6685649 recites a method for detection of nociception by analysis of RR intervals achieved either from ECG data or blood pressure data. From the RR intervals the acceleration emphasized RR interval is defined. Patent document US 2009/0076339 discloses a method for monitoring the nociception of a patient during general anesthesia by extracting RR intervals from ECG and blood pressure. The method is based on detection of simultaneous increase in HR and BP, defined as a non-baroreflex. However, the described method is not able to detect whether the patient has been overdosed with analgesia but rather only detect whether the patient responds or not to painful stimuli with a positive predictive value of 30%. In yet another example, patent document EP 1495715 recites a method for measuring an index of hypnosis as well as index of analgesia which are independent to each other.

Lastly, Marugg filed Oct. 12, 2013 “a device and method for determining the probability of response to pain and nociception of a subject t”. This is a method for determining the probability of response to pain and nociception (final qNOX) of a subject during different levels of arousal, comprising steps of: (a) receiving electroencephalography (EEG) data and electromyography (EMG) data; (b) defining an index of consciousness (qCON) as a function of the EEG data; (c) defining an index of nociception (initial qNOX) as a function of the EEG data and the EMG data; and, (d) defining a weighing factor alpha a as a function of qCON; wherein, if the initial qNOX>qCON and qCON<k1 a is defined by the following formula:[alpha]=k2-k4 * (qCON-k3); where k1 , k2, k3 and k4 are predetermined values; if [alpha]>k2, [alpha] is defined by the following formula [alpha]=k2; further wherein a final qNOX is defined by the following formula: final qNOX=(1−[alpha]) * initial qNOX+[alpha]* qCON. While this publication might be considered as the closest prior art, several features of the present invention are novel, not obvious and respond to a long felt need for a better and more complete apparatus for the assessment of the level of pain and nociception during general anesthesia.

### SUMMARY OF THE INVENTION

It is hence one object of the invention to disclose an apparatus equipped with means for estimating pain and nociception while awake, during general anesthesia or sedation from data including electroencephalogram (EEG), facial electromyogram (EMG), heart rate variability (HRV) by electrocardiogram (ECG) and plethysmography by impedance cardiography (ICG), the apparatus comprising the following features: means for obtaining a signal containing EEG and facial EMG, said mean adapted for recording from a subjects scalp with three electrodes positioned at middle forehead, left (right) forehead and the left (right) cheek; means for obtaining a three leads ECG signal and adaptations for calculating the R-R interval and the HRV from said ECG signal; means for obtaining an ICG signal with four electrodes positioned at the chest of the patient; means for obtaining the plethysmographic from the ICG; adaptation for calculating the Fast Fourier Transform (FFT) and the Choi-Williams distributions for about 1-60 seconds of the EEG signal; adaptations for calculating the frequency spectrum in about 1-10 seconds EMG signal; adaptations for calculating the spectral edge frequency of the spectrum of the R-R interval of the ECG over about 2-6 minutes; adaptations for calculating the Hilbert transform of the ICG signal from which the number of peaks over a certain threshold in the 1st derivative of the Hilbert phase, is estimated; means adapted for combining the extracted EEG, EMG, ECG, and ICG data into a final index of nociception represented by a scale from 0 to 99, where a value of 99 represents a high probability of response to light noxious stimuli and a value of close or equal to 0 corresponds to a total block of afferent noxious stimuli, for example obtained by local anesthetic drugs.

It is hence another object of the invention to disclose the apparatus further defined as the position of the electrodes can be either middle forehead (Fp, in the international 10-20 electrode positioning system), left forehead (F7) and the left cheek (temporal process) 2 cm below the middle eye line or the electrode position can alternatively be middle forehead, right forehead and the right cheek (temporal process) 2 cm below the middle eye line.

It is hence another object of the invention to disclose the apparatus further refined by calculating the HRV by doing a Fast Fourier Transform (FFT) of the signal defined as the R-R interval; the ninety-five percent Spectral Edge Frequency (SEF) of the spectrum is used for the subsequent calculation of the Index of nociception.

It is hence another object of the invention to disclose the apparatus further refined by calculating the Hilbert transform of the plethysmographic curve.

It is hence another object of the invention to disclose the apparatus further refined by estimating the Choi-Williams distribution over about at least 1-60 s and about at least 0-100 Hz, the variability of the energy of 1 s and 1 Hz squares are calculated.

It is hence another object of the invention to disclose the apparatus further refined by calculating the mutual information and Fokker-Planck drift and diffusion coefficients between the EEG and impedance cardiography.

It is hence another object of the invention to disclose the apparatus further defined by calculating the energy in different frequency bands of the spectrum.

It is hence another object of the invention to disclose the apparatus further defined as the principle of Fast Fourier Transform (FFT) of the R-R intervals; from the spectrum the ninety-five percent Spectral Edge Frequency (SEF) is calculated and used for the subsequent calculation of the Index of nociception.

It is hence another object of the invention to disclose the apparatus further refined as the ICG is measured with a sampling frequency of about 250 Hz whereafter the Hilbert transform is carried out on a signal of about 1 to 10 s duration; from which the number of peaks in the first derivative of the Hilbert Phase which are over a certain threshold, is calculated; this parameter is used for the subsequent calculation of the Index of nociception.

It is hence another object of the invention to disclose the apparatus that describe parameters which are all included in a classifier, which could be but not necessarily a multiple linear or logistic regression, a quadratic equation or a fuzzy inference reasoner or a an adaptive neuro fuzzy inference system, where the output of the classifier is the Index of nociception, an index associated with the probability of response of the patient to noxious stimuli.

It is hence one object of the invention to disclose a method for estimating pain and for calculating the level of nociception while awake, during general anesthesia or sedation from data including electroencephalogram (EEG), facial electromyogram (EMG), heart rate variability (HRV) by electrocardiogram (ECG) and plethysmography by impedance cardiography (ICG), the method comprising the steps of: obtaining a signal recording containing EEG and facial EMG from a subjects scalp with three electrodes positioned at middle forehead, left (right) forehead and the left (right) cheek; obtaining a three leads ECG signal and calculating the R-R interval and the HRV from said ECG signal; obtaining an ICG signal with four electrodes positioned at the chest of the patient; obtaining the plethysmographic from the ICG; calculating the Fast Fourier Transform (FFT) and the Choi-Williams distributions for about 1-60 seconds of the EEG signal; calculating the frequency spectrum in a 1-10 seconds EMG signal; calculating the spectral edge frequency of the spectrum of the R-R interval of the ECG over about 2-6 minutes; calculating the Hilbert transform of the ICG signal from which the number of peaks over a certain threshold in the 1st derivative of the Hilbert phase, is estimated; combining the extracted EEG, EMG, ECG, and ICG data into a final index of nociception represented by a scale from 0 to 99, where a value of 99 represents a high probability of response to light noxious stimuli and a value of close or equal to 0 corresponds to a total block of afferent noxious stimuli, for example obtained by local anesthetic drugs.

It is hence another object of the invention to disclose the method where said electrodes can be further positioned in a place selected from group consisting of: middle forehead (Fp, in the international 10-20 electrode positioning system), left forehead (F7) and the left cheek (temporal process) 2 cm below the middle eye line or the electrode position can alternatively be middle forehead, right forehead and the right cheek (temporal process) 2 cm below the middle eye line, and any combination thereof.

It is hence another object of the invention to disclose the method where said step further comprises a step of calculating the HRV by doing a Fast Fourier Transform (FFT) of the signal defined as the R-R interval; the ninety-five percent Spectral Edge Frequency (SEF) of the spectrum is used for the subsequent calculation of the Index of nociception.

It is hence another object of the invention to disclose the method where said step further comprises a step of calculating the Hilbert transform of the plethysmographic curve.

It is hence another object of the invention to disclose the method where said step further comprises a step of estimating the Choi-Williams distribution over about at least 1-60 s and about at least 0-100 Hz, the variability of the energy of 1 s and 1 Hz squares are calculated.

It is hence another object of the invention to disclose the method where said method further comprises a step of calculating the mutual information and Fokker-Planck drift and diffusion coefficients between the EEG and impedance cardiography.

It is hence another object of the invention to disclose the method where said step further comprises a step of calculating the energy in different frequency bands of the spectrum.

It is hence another object of the invention to disclose the method where said step is further defined as the principle of Fast Fourier Transform (FFT) of the R-R intervals; from the spectrum the ninety-five percent Spectral Edge Frequency (SEF) is calculated and used for the subsequent calculation of the Index of nociception.

It is hence another object of the invention to disclose the method where said step is further refined as the ICG is measured with a sampling frequency of about 250 Hz whereafter the Hilbert transform is carried out on a signal of 1 to 10 s duration; from which the number of peaks in the first derivative of the Hilbert phase which are over a certain threshold, is calculated; this parameter is used for the subsequent calculation of the Index of nociception.

It is hence another object of the invention to disclose the method where said method describe parameters which are all included in a classifier, which could be but not necessarily a multiple linear or logistic regression, quadratic equation or a fuzzy inference reasoner or a an adaptive neuro fuzzy inference system, where the output of the classifier is the Index of nociception, an index associated with the probability of response of the patient to noxious stimuli.

### DESCRIPTION OF THE INVENTION

The following description is provided, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide an apparatus for the assessment of the level of pain and nociception while awake or during general anesthesia. Thus a novel method for applying such apparatus has been obtained.

The term “EEG” refers herein after to electroencephalogram.

The term “EMG” refers hereinafter to facial electromyogram.

The term “HRV” refers hereinafter to heart rate variability.

The term “ECG” refers hereinafter to electrocardiogram.

The term “ICG” refers hereinafter to plethysmography by impedance cardiography.

The term “FFT” refers hereinafter to Fast Fourier Transform.

The term “CNI” refers hereinafter to Composite Nociception Index.

The term “ANFIS” refers hereinafter to Adaptive Neuro Fuzzy Inference System.

The term “SEF” refers hereinafter to Spectral Edge Frequency.

The term “about” refers hereinafter to ±25% of the mentioned value.

In one embodiment of the present invention the apparatus is equipped with means for measuring pain and adapted for calculating the level of nociception while awake, during general anesthesia or sedation from data including electroencephalogram (EEG), facial electromyogram (EMG), heart rate variability (HRV) by electrocardiogram (ECG) and plethysmography by impedance cardiography (ICG), the apparatus comprising the following features:
1. means for obtaining a signal containing EEG and facial EMG, said mean adapted for recording from a subjects scalp with three electrodes positioned at middle forehead, left (right) forehead and the left (right) cheek;
2. means for obtaining a three leads ECG signal and adaptations for calculating the R-R interval and the heart rate variability (HRV) from said ECG signal;
3. means for obtaining an impedance cardiography (ICG) signal with four electrodes positioned at the chest of the patient;
4. means for obtaining the plethysmographic from the impedance cardiography (ICG);
5. adaptation for calculating the Fast Fourier Transform (FFT) and the Choi-Williams distributions for 1-60 seconds of the EEG signal;
6. adaptations for calculating the frequency spectrum in a 1-10 seconds EMG signal;
7. adaptations for calculating the spectral edge frequency of the spectrum of the R-R interval of the ECG over approximately 2-6 minutes;
8. adaptations for calculating the Hilbert transform of the ICG signal from which the number of peaks over a certain threshold in the 1st derivative of the Hilbert phase, is estimated;
9. means adapted for combining the extracted EEG, EMG, ECG, and ICG data into a final index of nociception represented by a scale from 0 to 99, where a value of 99 represents a high probability of response to light noxious stimuli and a value of close or equal to 0 corresponds to a total block of afferent noxious stimuli, for example obtained by local anesthetic drugs.

Referring now to FIG. 1, showing the flow chart of the invention. The one channel EEG (2) and EMG (4) signals are recorded (1) from three surface electrodes positioned on the forehead of the subject. The ECG (8) is recorded with 4 surface electrodes positioned two on the chest and two on the neck, the Fast Fourier Transform (9) is estimated and subsequently the Heart Rate Variability is calculated (10). The parameters extracted from EEG, EMG, and HRV are all used as input to the classifier (13) which could be either a linear regression, a logistic regression, a fuzzy logic classifier, a neural network or a hybrid between a fuzzy logic system and a neural network such as an Adaptive Neuro Fuzzy Inference System (ANFIS). The output of the classifier is the Composite Nociception Index (CNI) (14). The CNI is assuming low values if the patient does not respond to noxious stimuli and high values if the patient is responding.

In one embodiment the position of the electrodes can be either middle forehead (Fp, in the international 10-20 electrode positioning system), left forehead (F7) and the left cheek (temporal process) 2 cm below the middle eye line or the electrode position can alternatively be middle forehead, right forehead and the right cheek (temporal process) 2 cm below the middle eye line.

The frequency bands (3) are extracted from the time domain of the EEG (2). An FFT is carried out of the EEG which enables the calculation of frequency ratios. The mutual information, Fokker-Planck drift and diffusion coefficients and cross correlation are calculated for the frequency bands of the EEG and ECG.

In the preferred embodiment the impedance cardiography is recorded from the same electrodes by which the ECG is recorded (6). A Hilbert transform and 1st derivative of the plethysmographic curve is used as input to the classifier (7).

An example of the behavior of the CNI is shown in FIG. 2. The x-axis denotes time while the y-axis denotes CNI and the effect site concentration of an analgesics, in this example remifentanyl. When no analgesics are administered to the patient, then the CNI is high, indicating that the patient has a high probability of responding to noxious stimuli, leading to hemodynamic complications such as myocardial infarction or stroke. When the analgetics, for example remifentanil, starts to take effect, then the CNI is dropping to a lower value, indicating less probability of response to a noxious stimulus. However, a very strong noxious stimulus might cause the CNI to increase although analgesia has been administered.

The range of the CNI could be from 0 to 99. The probability of response to noxious stimuli follows a sigmoidal shaped logistic regression. This is shown in FIG. 3 showing that a value of 99 of the CNI means that the probability of response to light noxious stimuli is close to 1. The definition of a CNI equal 0 is a total block of the afferent noxious stimulus.

The Heart Rate Variability

The HRV is calculated by performing a Fast Fourier Transform on the HR signal, over a 120-800 s period. From this, the Spectral Edge Frequency (SEF) is calculated as the frequency where 95% of the area of the total spectrum is obtained, as shown in FIG. 4. The SEF of the HRV is used as input to the classifier as shown in FIG. 1 (10).

The EEG

A Fast Fourier Transform (FFT) is applied to the EEG and the energy in frequency bands are defined. From that ratios are calculated which are used as input to the classifier. The mutual information, Fokker-Planck drift and diffusion coefficients and cross correlation are calculated for the frequencies calculated of the EEG, EMG and ECG.

The Plethysmographic Curve

In the preferred embodiment, the plethysmographic curve, is constructed by injection of a current in two electrodes, while voltage between two adjacent electrodes is recorded, preferably at a frequency in the 10-200 KHz range. The impedance is then calculated and the plethysmographic curve is then defined as the impedance or the impedance cardiography.

The Analgetics Concentration and Biophase

The concentration of the analgetics could be, but not limited to, the plasma concentration of the infused remifentanil by an infusion pump. The biophase could be the effect site concentration of remifentanil. Either could be used as input to the model.

In a preferred embodiment of the present invention the measurements abovementioned are performed while awake, during general anesthesia or sedation.

### BRIEF DESCRIPTION OF THE FIGURES

FIG. 1 showing a schematic flow chart of the present invention.

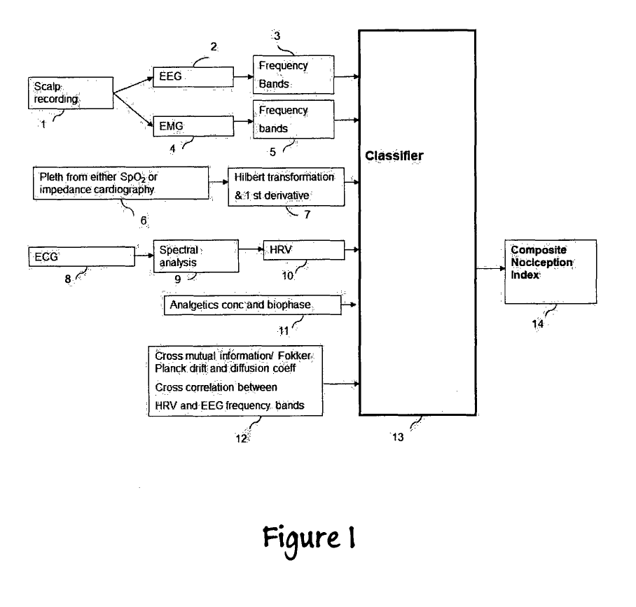

FIG. 2 showing a schematic graph of the evolution of the Composite Nociception Index (CNI) during infusion of an analgesic.

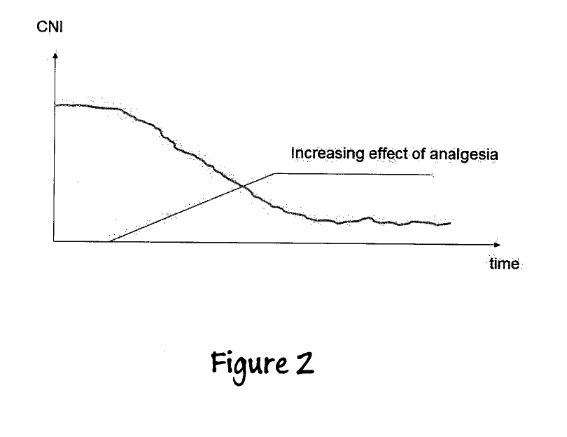

FIG. 3 showing a schematic graph of the sigmoidal curve showing the conceptual relationship between the Composite Nociception Index (CNI) and the probability of response to a noxious stimulus.

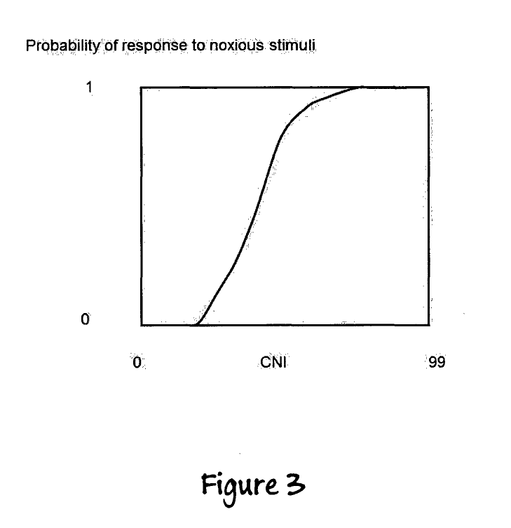

FIG. 4 showing a schematic flow chart of the spectral analysis of the R-R intervals which is estimated by a Fast Fourier transform. The Spectral Edge Frequency (SEF) is defined as the frequency where 95% of the area or energy of the spectrum is reached.

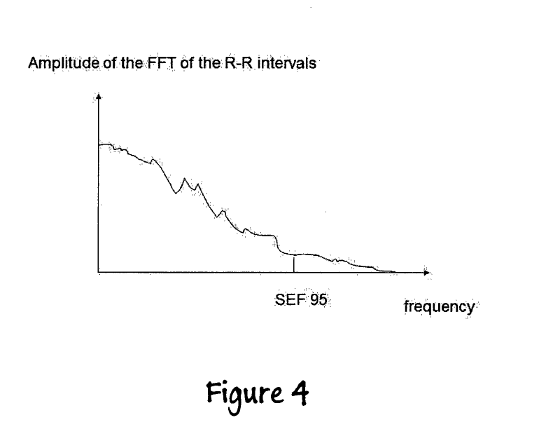

1. An apparatus equipped with means for estimating pain and nociception while awake, during general anesthesia or sedation from data including electroencephalogram (EEG), facial electromyogram (EMG), heart rate variability (HRV) by electrocardiogram (ECG) and plethysmography by impedance cardiography (ICG), the apparatus comprising the following features:
(a) means for obtaining a signal containing EEG and facial EMG, said mean adapted for recording from a subjects scalp with three electrodes positioned at middle forehead, left (right) forehead and the left (right) cheek;
(b) means for obtaining a three leads ECG signal and adaptations for calculating the R-R interval and the HRV from said ECG signal;
(c) means for obtaining an ICG signal with four electrodes positioned at the chest of the patient;
(d) means for obtaining the plethysmographic from the ICG;
(e) adaptation for calculating the Fast Fourier Transform (FFT) and the Choi-Williams distributions for about 1-60 seconds of the EEG signal;
(f) adaptations for calculating the frequency spectrum in about 1-10 seconds EMG signal;
(g) adaptations for calculating the spectral edge frequency of the spectrum of the R-R interval of the ECG over about 2-6 minutes;
(h) adaptations for calculating the Hilbert transform of the 1CG signal from which the number of peaks over a certain threshold in the 1st derivative of the Hilbert phase, is estimated;
(i) means adapted for combining the extracted EEG, EMG, EGG, and ICG data into a final index of nociception represented by a scale from 0 to 99, where a value of 99 represents a high probability of response to light noxious stimuli and a value of close or equal to 0 corresponds to a total block of afferent noxious stimuli, for example obtained by local anesthetic drugs.

2. The apparatus according to claim 1 is further defined as the position of the electrodes can be either middle forehead (Fp, in the international 10-20 electrode positioning system), left forehead (F7) and the left cheek (temporal process) 2 cm below the middle eye line or the electrode position can alternatively be middle forehead, right forehead and the right cheek (temporal process) 2 cm below the middle eye line.

3. The apparatus according to claim 1 is further refined by calculating the HRV by doing a Fast Fourier Transform (FFT) of the signal defined as the R-R interval and the ninety-five percent Spectral Edge Frequency (SEF) of the spectrum is used for a subsequent calculation of the Index of nociception.

4. The apparatus according to claim 1 is further refined by calculating the Hilbert transform of the plethysmographic curve.

5. The apparatus according to claim 1 is further refined by estimating the Choi-Williams distribution over about at least 1-60 s and about at least 0-100 Hz, the variability of the energy of 1 s and 1 Hz squares are calculated.

6. The apparatus according to claim 1 which is further refined by calculating the mutual information and Fokker-Planck drift and diffusion coefficients between the EEG and impedance cardiography.

7. The apparatus according to claim 1 is further defined by calculating the energy in different frequency bands of the spectrum.

8. The apparatus according to claim 1 is further defined as the principle of Fast Fourier Transform (FFT) of the R-R intervals; from the spectrum the ninety-five percent Spectral Edge Frequency (SEF) is calculated and used for the subsequent calculation of the Index of nociception.

9. The apparatus according to claim 1is further refined as the ICG is measured with a sampling frequency of about 250 Hz whereafter the Hilbert transform is carried out on a signal of about 1 to 10 s duration; from which the number of peaks in the first derivative of the Hilbert phase which are over a certain threshold, is calculated; this parameter is used for the subsequent calculation of the Index of nociception.

10. The apparatus according to claim 1 describe parameters which are all included in a classifier, which could be but not necessarily a multiple linear or logistic regression, a quadratic equation or a fuzzy inference reasoner or a an adaptive neuro fuzzy inference system, where the output of the classifier is the Index of nociception, an index associated with the probability of response of the patient to noxious stimuli.

11. A method for estimating pain and for calculating the level of nociception while awake, during general anesthesia or sedation from data including electroencephalogram (EEG), facial electromyogram (EMG), heart rate variability (HRV) by electrocardiogram (ECG) and plethysmography by impedance cardiography (ICG), the method comprising the steps of:
(a) obtaining a signal recording containing EEG and facial EMG from a subjects scalp with three electrodes positioned at middle forehead, left (right) forehead and the left (right) cheek;
(b) obtaining a three leads ECG signal and calculating the R-R interval and the HRV from said ECG signal;
(c) obtaining an ICG signal with four electrodes positioned at the chest of the patient;
(d) obtaining the plethysmographic from the ICG;
(e) calculating the Fast Fourier Transform (EFT) and the Choi-Williams distributions for about 1-60 seconds of the EEG signal;
(f) calculating the frequency spectrum in a 1-10 seconds EMG signal;
(g) calculating the spectral edge frequency of the spectrum of the R-R interval of the ECG over about 2-6 minutes;
(h) calculating the Hilbert transform of the ICG signal from which the number of peaks over a certain threshold in the 1st derivative of the Hilbert phase is estimated;
(i) combining the extracted EEG, EMG, ECG, and ICG data into a final index of nociception represented by a scale from 0 to 99, where a value of 99 represents a high probability of response to light noxious stimuli and a value of close or equal to 0 corresponds to a total block of afferent noxious stimuli, for example obtained by local anesthetic drugs.

12. The method according to claim 11, wherein said electrodes can be further positioned in a place selected from group consisting of: middle forehead (Fp, in the international 10-20 electrode positioning system), left forehead (F7) and the left cheek (temporal process) 2 cm below the middle eye line or the electrode position can alternatively be middle forehead, right forehead and the right cheek (temporal process) 2 cm below the middle eye line, and any combination thereof.

13. The method according to claim 11 further comprising a step of calculating the HRV by doing a Fast Fourier Transform (FFT) of the signal defined as the R-R interval; the ninety-five percent Spectral Edge Frequency (SEF) of the spectrum is used for the subsequent calculation of the Index of nociception.

14. The method according to claim 11 further comprising a step of calculating the Hilbert transform of the plethysmographic curve.

15. The method according to claim 11 further comprising a step of estimating the Choi-Williams distribution over about at least 1-60 s and about at least 0-100 Hz, the variability of the energy of 1 s and 1 Hz squares are calculated.

16. The method according to claim 11, wherein said method further comprises a step of calculating the mutual information and Fokker-Planck drift and diffusion coefficients between the EEG and impedance cardiography.

17. The method according to claim 11, wherein said step of calculating the frequency spectrum in a 1-10 seconds EMG signal further comprises a step of calculating the energy in different frequency bands of the spectrum.

18. The method according to claim 11, wherein said step of calculating the Hilbert transform of the ICG signal from which the number of peaks over a certain threshold in the 1^{st} derivative of the Hilbert phase is estimated is further defined as the principle of Fast Fourier Transform (FFT) of the R-R intervals;
from the spectrum the ninety-five percent Spectral Edge Frequency (SEF) is calculated and used for the subsequent calculation of the Index of nociception.

19. The method according to claim 11, wherein said step of calculating the Hilbert transform of the ICG signal from which the number of peaks over a certain threshold in the 1^{st} derivative of the Hilbert phase is estimated is further refined as the ICG is measured with a sampling frequency of about 10-1024 Hz whereafter the Hilbert transform is carried out on a signal of 1 to 10 s duration from which the number of peaks in the first derivative of the Hilbert phase which are over a certain threshold are calculated to yield a parameter that is used for a subsequent calculation of the Index of nociception.

20. The method according to claim 1, wherein said method describes parameters which are all included in a classifier, which could be but is not necessarily a multiple linear or logistic regression, quadratic equation or a fuzzy inference reasoner or a an adaptive neuro fuzzy inference system, where the output of the classifier is the Index of nociception, an index associated with the probability of response of the patient to noxious stimuli.
